(19) United States
(12) Patent Application Publication
(10) Pub. No.: US 2003/0129610 A1
(43) Pub. Date: Jul. 10, 2003

(54) New QTL's on chromosomes X, 2, 6 and 7 of pigs

(76) Inventors: Dirk-Jan de Koning, Renkum (NL); Martinus Antonius, M. Groenen, Zetten (NL); Johannus Antonius, M. van Arendonk, Bennekom (NL)

Correspondence Address: TRASK BRITT, P.O. BOX 2550, SALT LAKE CITY, UT, 84110, US

(21) Appl. No.: 10/177,252

(22) Filed: Jun. 21, 2002

Related U.S. Application Data
(63) Continuation of application No. PCT/NL00/00935, filed on Dec. 20, 2000.

(30) Foreign Application Priority Data
Dec. 21, 1999 (EP) 99204461.0

(51) Int. Cl. C12Q 1/68; C07H 21/04; C12P 21/02; C12N 5/06; C07K 14/74

(52) U.S. Cl. 435/6; 435/69.1; 435/320.1; 435/325; 530/350; 536/23.5

(57) ABSTRACT

The present invention relates to the field of breeding domestic animals, particularly pigs. In particular, it relates to genotypic and/or phenotypic traits that need to be selected for in-breeding animals or animals intended for slaughter. The invention provides for the use of well-defined imprinted and x-linked markers for quality directed crossbreeding in pig breeding programs, in particular the use of new QTL's or markers on chromosomes X, 2, 6 and 7 of pigs is provided.

### CROSS-REFERENCE TO RELATED APPLICATION

This application is a continuation of International Application Number PCT/NL00/00935 filed on Dec. 20, 2000 designating the United States of America, International Publication No. WO 01/46406 (Jun. 28, 2001), the contents of the entirety of which is incorporated by this reference.

### TECHNICAL FIELD

The present invention relates to the field of breeding domestic animals, in particular pigs. In particular it relates to genotypic and/or phenotypic traits which need to be selected for breeding animals or animals intended for slaughter. Traits having to do with desired phenotypic properties in domestic animals, such as back fat, muscle depth, growth rate and feed conversion are often referred to as quantitative traits. In recent years efforts have been directed at identifying the genomic localization of such quantitative traits, e.g., by means of microsatellite markers. The resulting localization leads to identification of so-called quantitative trait loci (QTL). Identifying QTL is an important part of arriving at means for selecting suitable breeding animals having desired genotypic and/or phenotypic properties, but also very important is determining the way of inheritance of such traits into offspring. The present invention provides a number of new QTL and also provides information on their inheritance pattern, thus providing new ways for selecting animals for breeding purposes and/or slaughtering and for implementing marker assisted breeding methods.

Thus, the present invention provides an isolated and/or recombinant nucleic acid comprising a sequence affecting the quantitative trait muscle depth in mammals, said sequence being derived from a locus corresponding to region HSA6p21.3-p.22 in humans and corresponding to the homologous region on ssc7 in pigs, wherein said quantitative trait locus is maternally expressed.

The invention further provides an isolated and/or recombinant nucleic acid comprising a sequence affecting the quantitative trait back fat thickness in mammals, said sequence being derived from a locus corresponding to a region on ssc7 in pigs, which region overlaps with the region identified in claim 1 affecting the quantitative trait muscle depth on ssc7 in pigs. This QTL inherits in a Mendelian fashion.

In yet another embodiment the invention provides an isolated and/or recombinant nucleic acid comprising a sequence affecting the quantitative trait backfat thickness in mammals, said sequence being derived from a locus corresponding to a region on ssc2 of pigs which maps about 35 cM away from the IGF2 region, wherein said quantitative trait locus is paternally expressed.

In another embodiment the invention provides an isolated and/or recombinant nucleic acid comprising the sequence of a maternally expressed quantitative trait locus affecting intramuscular fat in mammals, said sequence being derived from a locus corresponding to a region on the long arm of ssc6 of pigs.

In yet another embodiment the invention provides an isolated and/or recombinant nucleic acid comprising a sequence of the paternally expressed quantitative trait locus affecting intramuscular fat in mammals, said sequence being derived from a region HSA6q22-ter in humans and corresponding to the homologous on the short arm of ssc6 of pigs.

In yet another embodiment the invention provides an isolated and/or recombinant nucleic acid comprising a sequence affecting the quantitative trait intramusccular fat in mammals, said sequence being derived from a region localised at about 60 cM on the X chromosome in pigs, and also an isolated and/or recombinant nucleic acid comprising a sequence affecting the quantitative trait back fat thickness in mammals, said sequence being derived from a region localised at about 69 cM on the X chromosome in pigs.

The recombinant nucleic acids according to the invention can be used in breeding programs in the same manner, QTL's have been used in selection methods so far. Care should be taken however of the fashion in which the traits inherit. Combining Mendelian traits and imprinted traits will give rise to different selections and breeding programmes than relying on either one of these. It provides tools to arrive at improved properties in an easier, more reproducible way. The QTL's according to the invention affect a number of similar traits and can thus be ideally combined for just such a purpose. The nucleic acids according to the invention can of course also be applied in the production of transgenic animals if such is desired.

Furthermore based on the identified QTL's isolated nucleic acids can be (synthetically or recombinantly) prepared to be used to detect these traits in animals or samples from animals, in particular mammals, especially pigs. For detecting such traits using any hybridisation technique, including amplification, in situ hybridisation and the like, such isolated nucleic acids can be used. They may be labelled for such a purpose, during, before or after the hybridisation step, in order to allow for easy detection. Thus the invention also provides an isolated and/or recombinant nucleic acid according to the invention which is a specific probe or primer for a quantitative trait and/or an isolated and/or recombinant nucleic acid comprising a sequence capable of identifying a quantitative trait as identified herein before. Typically such a nucleic acid may be based on, or derived from a microsatellite marker, that is located near the actual QTL. The invention of course also provides the use of an isolated and/or recombinant nucleic acid according to the invention in identifying a quantitative trait locus in a mammal, in particular also when said quantitative trait locus is parentally imprinted.

The invention also provides the use of an isolated and/or recombinant nucleic acid acoording to the invention for selection of a domestic animal having desired genetic properties. Taking a sample from an animal and testing it with a nucleic acid according to the invention is well within the skill of the art and needs no further explanation here. Selection of the desired allele is also within the art, e.g. using mismatch PCR and other techniques for distinguishing alleles.

The invention also includes these uses wherein said domestic animal is a breeding animal or an animal for slaughter. Of course the results of testing in particular breeding animals for desired properties will be put to, good use in breeding programmes. Thus the invention also includes methods for breeding a population of domestic animals having desired genetic properties, comprising testing animals to be cross-bred with at least one nucleic acid according to the invention, for the presence of a desired allele of a quantitative trait, in particular where such a method comprises a method wherein said testing comprises contacting a sample from an animal to be cross-bred with a nucleic acid according the invention and detecting the presence or absence of hybridisation. Cells and animals provided with a nucleic acid according to the invention are also provided by the present invention. Thus the invention provides a mammalian cell comprising a recombinant nucleic acid corresponding to a QTL as defined according to the invention, as well as a transgenic animal comprising at least one cell as disclosed above.

### DETAILED DESCRIPTION

Recently imprinting at the IGF2 locus in two different pig crosses^{1,2}, was disclosed. We have investigated the role of imprinting in an intercross between Chinese Meishan and commercial Dutch pigs. On 785 F₂ animals we recorded three body composition traits after slaughter: backfat thickness (BFT), muscle depth (MD), and the percentage of intramuscular fat (IMF) inside the Musculus longissimus³. An earlier analysis⁴ of BFT and IMF showed significant evidence for QTLs on chromosomes 2 and 7, ignoring effects of imprinting.

A whole genome scan using 132 microsatellite markers was used-to map autosomal QTLs on the F₂ population, including a test for imprinting. The statistical model for imprinting⁵ was re-parameterized to separate the contribution of the paternally and maternally inherited effect. Mode of inheritance for a putative QTL was inferred from the variance explained by the components in the model. Our genome scan resulted in five significant QTLs affecting body composition traits, of which four were imprinted.

There was strong evidence for a paternally expressed QTL for BFT on chromosome 2 (Table 1). For the QTL affecting BFT on chromosome 7, both the paternal and maternal component were highly significant implying Mendelian expression for this QTL. Also on chromosome 7, a highly significant QTL for MD mapped to the same area as the QTL for BFT. In contrast to the QTL for BFT, however, the QTL for MD was maternally expressed (Table 1). From these results, it cannot be distinguished whether there are two separate loci or one pleiotropic locus that shows imprinting during one stage of development and Mendelian expression during another.

With a model ignoring imprinting³, suggestive evidence for a Mendelian QTL for IMF was found on the long arm of chromosome 6. The present analysis, however, revealed that this was caused by a paternally expressed QTL (Table 1). In addition, a maternally expressed QTL was found on the short arm of the same chromosome.

A graphical comparison of results obtained under the imprinting and Mendelian models is given in FIG. 1. The imprinted QTL on chromosome 2 maps at about 35 cM away from the IGF2 region, for which there is an imprinted QTL for body composition¹

The general outline of the comparative map between pig and human for the regions of interest^{6,7} has been established and is shown in FIG. 1. Genes that have been mapped more precisely in the pig by linkage analysis or on the radiation hybrid panel⁹ facilitated refinement of the comparative map. We realize that the comparative map presented here is not comprehensive, and that some genes originating from other chromosomes are reported but not represented in FIG. 1.

The QTL for MD on SSC7 can be narrowed to a region homologous with HSA6p21.3-p22. This region contains the major histocompatibility complex including LTA and shows extensive conservation in gene order¹⁰. Imprinted genes have not been reported for this region in humans and mice¹¹ (http://www.mgc.har.mrc.ac.uk/imprinting/imptables.html). Several genes that map to the area of the maternally expressed QTL on SSC6p are located on HSA 16q22-ter, where imprinting has not been reported either. The area within the confidence interval of the paternally expressed QTL for IMF on SSC6q, however, extends on both sides to homologous regions in humans, where imprinted genes have been reported (PEG3 on HSA 19q13.4 is imprinted in mice and p73 on HSA 1p36 is maternally expressed). Genes MC5R, FABP3¹² and UOX⁹ within the interval SW316-S0003 covering the peak of the QTL, are located on HSA18p11.2, 1p33-p32, and 1p22, respectively. Imprinting has not been found for these candidate regions in humans. For chromosome 2, imprinting is reported for the IGF2 area but until now homology to other imprinting clusters cannot be established clearly. Data on imprinting of Wilms Tumor 1 (WT1) on HSA11p13 demonstrate that imprinting can be specific for certain tissues and developmental stages¹¹

The X chromosome harbors quantitative trait loci for backfat thickness and intramuscular fat content in pigs

Genetic markers have been used in experimental crosses in pigs to dissect quantitative trait variation (e.g. Andersson et al. 1994, Knott et al. 1998, Rohrer and Keele, 1998, Walling et al. 1998). We reported recently on the mapping of autosomal quantitative trait loci (QTL) for fatness traits in an experimental intercross between the obese Chinese Meishan breed and Dutch White production lines^{4,15}. In this paper, the analysis of the X chromosome on the total population described by Janss et al. (1997) is presented.

Briefly, 19 Meishan boars were mated to 120 sows of five different Large White and Landrace lines from Dutch breeding companies. From the F1 animals, 39 F1 and 124 F1 sows were randomly selected to generate the F2 generation. The F2 animals (n=1293), their F1 parents (n=303) and the 19 Meishan grandfathers were genotyped for 5 microsateHite markers located in the non-pseudoautosomal region of the X chromosome. The amount of fat within the muscle (intramuscular fat IMF) and backfat thickness (BFT) of the musculus longissimus was recorded on 785 F2 animals after slaughter at a live weight of approximately 90 kg³. For QTL analysis, interval mapping by regression was carried out as a line cross analysis as described previously⁴ where the founder lines were assumed to be fixed for different QTL alleles. The model was modified to account for differences between sexes. According to the design of the cross, male F2 offspring carry only a maternal copy of the X chromosome originating from the Meishan or from the White grandparents, whereas all female offspring inherited in addition the paternal copy of the X chromosome originating from the White lines. Therefore, the contrast between the Meishan and the White allele for the estimation of the QTL effect was estimated within male and female F2 offspring separately.

The female genetic map calculated from our data (FIG. 2) shows the same marker order but differences in distances between markers compared to the map of Rohrer et al. (1996). An increase in length of roughly 10 cM (Kosambi) is observed between markers SW2534 and SW2456 (37.8 cM versus 27.6 cM) as well as between SW2476 and SW1943 (21.5 cM versus 9.8 cM). However, a smaller distance is observed on our map in the interval between SW2456 and SW2476 (9.4 cM versus 19.8 cM). Sensitivity analysis did not point towards specific families causing these differences (data not shown). As the mapping population of Rohrer et al. (1996) consisted of 94 F2 animals and only female parents contribute mapping information, the recombination frequencies are expected to be rather rough estimates and might explain the differences observed.

FIG. 2 shows the test statistics of the QTL analysis for IMF and BFT and the threshold levels along the genetic map of the X chromosome. For both fatness traits, a genomewise significant (p<5%) QTL is detected. The estimated best position of the QTLs is in different but neighbouring marker intervals at 60 cM for BFT and 69 cM for IMF (Table 2). As expected, the alleles from the obese Meishan breed increase the amount of intramuscular fat as well as back fat. The estimated size of the QTL effect is smaller in females than in males for both traits (Table 2).

In summary, this research reveals a number of imprinted and X-linked QTL's and at least two chromosomal areas to which imprinted genes had so far not been mapped in pigs, and the known homologies to humans and mice do not reveal obvious positional imprinted candidates. Results indicate new areas subject to imprinting that affect body composition. Although obesity genes reported in humans and mice map to homologous regions of the imprinted QTLs found in this study¹³, imprinting has only been reported for the Prader-Willi Syndrome (HSA15q11.2-q12). According to our study, imprinting might be more common in development of obesity than currently anticipated.

For the practice of animal breeding, identification of major imprinted loci affecting body composition has several implications. Our results call for a revision of the breeding evaluation methods that are based on the assumption of a large number of genes acting additively. Identification of imprinted loci opens new perspectives for crossbreeding which is common practice in pig breeding. Imprinted genes could further accommodate the differentiation between sow lines, which are required to have optimal body composition to support their reproductive performance, and boar lines, which ensure high quality pork.

Although the mechanisms underlying imprinting are not totally unravelled¹⁴, this study clearly demonstrates the important role of imprinting for body composition traits. Statistical testing for imprinting should become a standard procedure in human and animal genetic research, both in genome scans and in evaluating candidate genes. The invention for example finds its use in present pig breeding programmes with pure breeding for additive genetic progress and crossbreeding for crossbreeding (dominance) effects (heterosis) in the final breeding product, are based on mendelian inheritance. Generally, in sire lines selection is focussed on production and meat quality traits like daily gain, backfat thickness (BF) muscle depth (MD) and intramuscular fat (IMF), while in dam lines selection is focussed on fertility traits.

Selection is only on fertility in the dam lines because the genetic background for fertility is mainly relevant in CD sows (90% of total sow population). Selection for daily gain, backfat and carcass traits is mainly performed in sire lines. Due to short generation interval in these lines, additive genetic progress is best reachable in these lines. Although the slaughter pig receives the genes for traits like backfat thickness and daily gain both from its dam and sire, selection for these traits is limited in dam lines because of negative genetic relationships between fertility and production traits.

The use of parental imprinting will change the set up of pig breeding programmes and improve its efficiency enormously.

New and economically relevant applications of parental imprinted markers/genes for BF and IMF are: for example shown in FIG. 3A.

1. Paternally expressed markers/genes for BF enable the possibility to select a dam (eg C) line for thick backfat, with expression of these genes in the CD-sows but without expression of these genes in the slaughter pig. By this procedure the favourable biological relationship between fertility and backfat can be used without having ‘fat’ slaughterpigs.

2. Paternally expressed markers/genes for IMF enable also the set up of a sire line (eg A) with a high genetic level of IMF but with 2 different applications:

a) the use of the sire line as a purebred boar (A in FIG. 3B) will result in slaughter pigs with high IMF

b) the use of sire line as the dam of a crossbred boar (B×A in FIG. 3A) will result in slaughter pigs without the high IMF-level.

The invention is in general applicable to purebred sire lines selection on paternally expressed genes/markers which need to be expressed in the slaughter pig and to purebred dam lines selection on maternal expressed genes/markers which need to be expressed in the slaughter pig.

### REFERENCES

1. Nezer, C. et al. Nature Genet. 21, 155-156 (1999)

2. Jeon, J. -T. et al. Nature Genet. 21, 157-158 (1999).

3. Janss, L. L. G., Van Arendonk, J. A. M. & Brascamp, E. W. Genetics 145, 395-408 (1997).

4. De Koning, D. J. et al. Genetics 152, 1679-1690 (1999).

5. Knott, S. A. et al. Genetics 149, 1069-1080 (1998).

6. Goureau, A. et al. Genomics 36, 252-262 (1996).

7. Rettenberger, G. et al. Genomics 26, 372-378 (1995).

8. Pearsall, R. S. et al. Mamm. Genome 9, 261-262 (1998).

9. Hawken, R. J. et al. Mamm. Genome 10, 824-830 (1999).

10. Chardon, P., Renard, C. & Vaiman, M. Immunol. Rev. 167, 179-192 (1999).

11. Morison, I. M. & Reeve, A. E. Hum. Mol. Genet. 7, 1599-1609 (1998).

12. Gerbens, F., Rettenberger, G., Lenstra, J. A., Veerkamp, J. H. & Te Pas, M. F. Mamm. Genome 8, 328-332 (1997).

13. Perusse, L. et al. Obesity Res. 7, 111-129 (1999).

14. Constancia, M., Pickard, B., Kelsey, G. & Reik, W. Genome Res. 8, 881-900 (1998).

15. Rattink et al., 1999

Further Examples

In pig breeding, strong selection pressure is applied to growth and reproduction traits. Using modern molecular technology it has been possible to identify QTL affecting growth, fatness, and litter size by scanning the entire genome (e.g. Andersson et al., 1994; Rohrer and Keele, 1998; Rohrer et al., 1999) or using a candidate gene approach (Rothschild et al., 1996; Kim et al., 2000). Genome scans are often performed on crosses between genetically distant pig breeds and analyzed under the line-cross model proposed by Haley et al. (1994). Knott et al. (1998) introduced extensions to this model to test for sex specific QTL effects, QTL on the X chromosome, and genomic imprinting effects. Using only phenotypic data, De Vries et al. (1994) were the first to show that genomic imprinting may influence the rate and composition of growth in pigs. Recently this was corroborated by a report of an imprinted QTL affecting fatness on SSC4 (Knott et al., 1998) and an imprinted QTL affecting muscularity and fatness in pigs in the IGF2 region on SSC2 (Jeon et al., 1999; Nezer et al., 1999). Rohrer and Keele (1998) described a QTL affecting backfat thickness on the X chromosome.

The present study describes the location of QTL that affect growth rate, backfat thickness, intramuscular fat and litter size using animals of an experimental cross between Meishan and Dutch commercial lines. The traditional line-cross analyses were complemented with systematic tests for imprinting and sex specific QTL effects. The production traits were also analyzed under a half-sib model in order to investigate the segregation of QTL alleles and effects within the F1. A genome scan was performed on F2 animals of a cross between Meishan and Dutch commercial lines. Phenotypic data was available for growth traits, ultrasonic backfat thickness and meat quality traits on 942-1151 animals and for litter size at two parities for 249 and 206 animals, respectively. QTL analyses were performed using interval mapping by regression under the line-cross approach complemented by tests for genomic imprinting and sex-specific QTL effects. For backfat thickness, the analyses revealed significant QTL on chromosomes 2, 7, 14, and X, with significant imprinting for chromosomes 2 and 14. For the different growth traits, significant QTL were detected on chromosomes 1, 4, 7, and 8. Both the QTL on chromosome 4 and chromosome 8 showed maternal expression for a specific growth stage. The QTL analyses for litter size revealed one suggestive QTL for first parity and three suggestive QTL for the second parity. Analyses under a half-sib model did not reveal additional significant QTL, but confirmed several of the QTL detected under the line-cross models. This study provides confirmation of several QTL affecting growth, fat deposition and meat quality in pigs and adds interesting new insight into their mode of expression.

2. Materials and Methods

2.1. Population, Phenotypes, and Markers

An F2 cross between the Chinese Meishan pig breed and commercial Dutch pig lines was available from an experiment involving five Dutch pig breeding companies, which has been described in detail by Janss et al. (1997). An F1 was obtained by artificial insemination of purebred sows from Large White and Dutch Landrace lines with semen from 19 boars from the Meishan breed. From the F1, >250 sows and 38 boars were randomly selected to become parents of the F2 litters. F2 animals were performance tested and three growth traits were defined: 1) early growth (EGR); daily gain from weaning to approximately 25 kg. 2) test growth (TGR); daily gain from approximately 25 to 90 kg. and 3) life growth (LGR); daily gain during entire life, not adjusting for birth weight. At the end of the performance test, backfat thickness (BFT) was measured ultrasonically and averaged over 4-8 measurements along the spine. A selection of F2 sows was inseminated with boars from the breeding company where they were kept and litter size, including stillborn piglets, was recorded at two parities (LS1 and LS2). An overview of the traits is given in Table 3.

Prior to the QTL analyses, the phenotypic data were adjusted for a number of systematic effects following Janss et al. (1997). The phenotypic data were analyzed under a polygenic inheritance model containing a fixed effect of period by company for all traits, an additional sex by company effect for the performance traits, and body weight at end of test as a covariable for BFT. These estimations were performed using the MAGGIC software package developed by Janss et al. (1995). Janss et al (1997) included phenotypic information on the F1 animals in the analysis whereas the current analyses were only based on phenotypes from the F2.

The F2 animals, their F1 parents and the F0 Meishan sires were typed for 132 microsatellite markers. The number of markers per chromosome varies between 15 markers on SSC2 and two on SSC18. Details on laboratory protocols and map construction can be found from De Koning et al. (1999).

2.2 QTL Analysis

For all traits interval mapping, using regression methods, was applied following the line-cross approach proposed by Haley et al. (1994). Under the line-cross model it is assumed that the two founder lines are fixed for alternative alleles at the QTL affecting the traits of interest, although they may share alleles at the marker loci. Using multi-marker information, four probabilities are calculated at 1 cM intervals along the genome. P₁₁ is the probability are that an F2 animal inherited two Meishan alleles, p₂₂ that it inherited two Dutch alleles, and p₁₂ or p₂₁ that it inherited one from each line (different subscripts according to parental origin; first subscript is paternally inherited allele). At every cM across the genome, an additive effect (a) and a dominance effect (d) are estimated using the regression of the phenotypes on a linear combination of the line origin probabilities:

y_{a,}=m+ap_{a,}+dp_{a,}+e_{a,} (1)

Where y_{a,} is the trait score of animal j (adjusted for systematic effects), m is the population mean, a and d are the estimated additive and dominant effect of a putative QTL at the given location, p_{a,} is the conditional probability of animal j to carry two Meishan alleles (p₁₁-p₂₂), pdj the conditional probability of animal j to be heterozygous (p₁₂+p₂₁), and ej is the residual error. A detailed description of these methods is given by Haley et al. (1994) and applications to crossbred pig populations are numerous (e.g. Andersson et al., 1994; Knott et al., 1998).

The line-cross analyses were extended with a test for sex-specific QTL effects following Knott et al. (1998). The model with sex specific QTL effects was accepted if the F test against the model with equal effects for both sexes was significant (p<0.05).

Imprinting was tested following the procedures presented by De Koning et al. (2000). To contribution of the parents was separated using the probability that the individual inherited a Meishan allele from its father (pₚₐₜ=[p₁₁+p₁₂]−[p₂₂+p₂₁]) or from its mother (pₘₐₜ=[p₁₁+p₂₁]−[p₂₂+p₁₂]). This re-parameterization allowed additional models to be fitted with exclusive paternal or maternal expression. All putative QTL locations from the three models were subsequently evaluated with a saturated model that contained a paternal, maternal and dominance component:

y_{a,}=m+aₚₐₜPₚₐₜⱼ+aₘₐₜPₘₐₜⱼ+dp_{a,}+e_{a,} (2)

Using F ratios for the individual components of the model, imprinting was inferred if only one of the parental contributions was significant and no dominance was present.

The X chromosome was analyzed under the line-cross model as described by Knott et al. (1998) and implemented for this experimental population by Harlizius et al. (2000). The analyses accounted for the non-reciprocal nature of the F2 cross and differences in probabilities for recombination between a marker and a putative QTL compared to the autosomes. QTL effects on the X chromosome were estimated separately within F2 males and females for the performance traits.

QTL analyses were also performed under a paternal half-sib model (Knott et al., 1996). However, half-sib family sizes were too small for litter size so these analyses were only carried out for the performance traits. The half-sib model makes no assumption on the number of QTL alleles and allele frequencies within the founder lines. For the half-sib analysis, the F2 animals are treated as 38 unrelated half-sib families and contrasts are made between the two haplotypes of every F1 boar. Within every half-sib family a QTL with a gene substitution effect is fitted at 1 cM intervals along the chromosome:

y_{a,}=m_{a,}+bᵢpᵢⱼ+e_{a,} (3)

Where yᵢⱼ is the adjusted trait score of individual j, originating from boar i; mi is the average effect for half-sib family i; bi is the substitution effect for a putative QTL; pij is the conditional probability for individual j of inheriting the first paternal haplotype; and eij is the residual effect. The test statistic is calculated as an F ratio for every map position within and across families. For details on half-sib analyses applied to this experimental population see De Koning et al. (1999). In the families that were inferred to be segregating for an identified QTL it was determined whether the Meishan allele was associated with an increase or a decrease in phenotype.

Significance of QTL was evaluated using two thresholds. The first level was suggestive linkage where one false positive is expected in a genome scan (Lander and Kruglyak, 1995). The suggestive significance level is proportional to the contribution (r) of a specific chromosome to the total genome length, which was obtained by dividing the length of a chromosome by the total length of the genome. For claiming significant linkage, the more stringent 5% genome-wide significance level was used. Both significance levels do not take the testing of multiple traits and models in the present and future studies into account.

Although calculated as an F ratio, the distribution of the test statistic under the H0 of no QTL is unknown for both the line-cross and half-sib analyses. Therefore, significance thresholds were determined empirically by permutation for individual chromosomes (Churchill and Doerge, 1994). To derive genome-wide significance levels from these chromosome-wide significance levels, the following Bonferoni correction was applied.

P_{genome-wide}=1−(1−p_{chromosome-wide})^{1/r} (4)

The test statistics for the different models have different degrees of freedom. Furthermore, there are differences in number of animals per trait, which further complicates comparisons between different analyses. As a result the empirical genome-wide significance threshold for the test statistic varied between ˜2.0 for the half-sib analyses and ˜12.5 for an imprinted QTL under the line-cross analyses. To facilitate graphical comparisons of different models, a transformation was applied to the test statistics. The tabulated p value was calculated for every test statistic, using an F distribution with the appropriate degrees of freedom. In the graphs, the negative logarithm of these p values is presented [−¹⁰LOG(p)]. Applying this transformation, the empirical threshold levels varied between 2.0 and 2.1 for suggestive linkage and between 3.4 and 3.7 for significant linkage, across all chromosomes, models and traits. Within the same trait or chromosome the range was even smaller. In the graphs, the average threshold over all traits and models that are represented in a specific graph was used.

3. Results

3.1 Results for Performance Traits

For the performance traits, the line-cross analyses revealed 12 genome-wide significant QTL and 25 suggestive QTL (Table 4). The half-sib analyses revealed five genome-wide significant, and three suggestive QTL (Table 5). For each of the performance traits, the highly suggestive and significant QTL will be discussed in the following paragraphs.

3.1.1. Results for EGR

For EGR, genome-wide significant QTL were detected on SSC4 and SSC8. The QTL on SSC4 showed significant differences in QTL effects between the sexes, with opposite sign for the additive effect and larger estimates for both the additive and dominance effect in the F2 females (Table 4). A graph of the test statistics of all QTL on SSC4 that were detected in the present study is given in FIG. 4. The QTL on SSC8 was maternally expressed with an estimated difference of 23 g/day lower growth for an animal with a maternally inherited Meishan allele compared to that with a maternal allele from the Dutch lines. An overview of the test statistic for the maternally expressed QTL affecting growth on SSC8 is given in FIG. 5.

Four highly suggestive QTL, with genome-wide p values between 0.06 and 0.07 were detected on SSC1, SSC6, SSC10, and an additional region on SSC4 (Table 4, FIG. 4). The suggestive QTL on SSC1 shows sex specific QTL effects with a larger effect in the F2 males (Table 4, FIG. 6). The suggestive QTL on SSC6 and SSC10 are imprinted, with exclusive maternal and paternal expression, respectively. Further suggestive evidence was obtained for a paternally expressed QTL at the end of SSC1, an over-dominant QTL on SSC5, a QTL with opposite dominance effects between the sexes on SSC8, and a maternally expressed QTL on SSC13 (Table 4). For all putative QTL affecting EGR the Meishan allele gives rise to lower growth except for the suggestive imprinted QTL on SSC1 where a paternally inherited Meishan allele gives rise to higher growth. The half-sib analyses showed no evidence for QTL affecting EGR.

3.1.2. Results for TGR

The line-cross analyses revealed genome-wide significant QTL affecting TGR on SSC1, SSC4, and SSC7 (Table 4). The QTL affecting TGR on SSC1 maps to a different region than both putative QTL affecting EGR on SSC1 (FIG. 6). This QTL affects TGR mainly additive with an estimated additive effect of −26.2 g/day. The significant QTL on SSC4 maps to the same position as the highly suggestive QTL affecting EGR, but for TGR this QTL is imprinted with exclusive maternal expression (FIG. 4). The most significant QTL was detected near the SLA region on SSC7. The estimated additive effect of 40.8 g/day indicates higher growth for a Meishan allele compared to an allele of the Dutch lines. This QTL was genome-wide significant under the half-sib model (Table 5), where nine families exceeded the nominal 5% threshold. These families show allele substitution effects between 76 and 155 g/day with higher growth for the Meishan allele in all these families. The line-cross analyses showed six suggestive QTL affecting TGR. Of these, paternal expression was inferred for the suggestive QTL on SSC2, SSC6, SSC12, and SSC14. A suggestive QTL on SSC8 was maternally expressed but mapped to a different region than the maternally expressed QTL affecting EGR on SSC8 (FIG. 5). The strongest suggestive QTL mapped to SSC13 and showed strong over-dominance (Table 4). For all putative QTL affecting TGR the Meishan allele is associated with lower growth, apart from the QTL on SSC7 Table 4.

3.1.3. Results for LGR

Under the line-cross analyses, three significant QTL affecting LGR were found on SSC1, SSC4, and SSC 7. Furthermore, eight suggestive QTL were detected on SSC3 (2), SSC5, SSC6 (2), SSC8, SSC13, and SSC17 (Table 4). The half-sib analyses showed significant QTL affecting LGR on SSCI and SSC7, and suggestive QTL on SSC3 and SSC6 (Table 5).

Under the line-cross model, the significant QTL affecting LGR on SSC1 maps at 38 cM, exactly between the sex specific QTL affecting EGR and the QTL affecting TGR (FIG. 6). A 5 cM grid search, fitting two QTL simultaneously, resulted in two QTL affecting LGR at 16 and 71 cM, but this was no significant improvement compared to fitting the single best QTL at 38 cM. Under the half-sib approach, a significant QTL on SSC1 mapped to the same interval as the QTL for TGR under the line-cross model (Table 4, Table 5, and FIG. 6). Estimated allele substitution effects at this position for seven informative families varied between 50 and 100 g/day. For five families it was derived that the Meishan gave lower growth whereas for one family the Meishan allele gave higher growth.

The QTL affecting LGR on SSC4 mapped to the same region as those detected for EGR and TGR (FIG. 4). The estimated additive and dominance effects are comparable to those of the Mendelian QTL affecting EGR.

The QTL on SSC7 is similar to that detected for TGR, with a positive effect on growth for the Meishan allele, albeit smaller than the effect for TGR (Table 4). Under the half-sib model, the estimated effects for 13 informative families are also smaller compared to TGR: between 52 and 102 g/day. Within these families the Meishan allele was consistently associated with lower growth.

A highly suggestive, maternally expressed QTL (p=0.07) mapped to the neighboring interval of the maternally expressed QTL affecting EGR on SSC8 (Table 4, FIG. 5). On SSC3, a suggestive QTL is detected at the beginning of the linkage group under both the line-cross (paternally expressed) and the half-sib approach, with an additional suggestive QTL at 20 cM under the line-cross approach. For SSC6, the line-cross model showed a maternally expressed QTL at 7 cM and a Mendelian QTL at 33 cM, the latter coinciding with a suggestive QTL affecting TGR (Table 4). In contrast, the half-sib analysis showed a suggestive QTL at 191 cM of SSC6 (Table 5). The suggestive QTL affecting LGR on SSC13 mapped to the same area as the suggestive QTL affecting TGR and was also over-dominant.

3.1.4. Results for BFT

The line-cross analyses showed significant QTL affecting BFT on SSC2, SSC7, SSC14, and the X chromosome. Additional suggestive QTL were detected on SSC4, SSC5, and SSC6 (Table 4). Under the half-sib analyses significant QTL were detected on SSC2 and SSC7 as well as a suggestive QTL on SSC14.

The highly significant QTL on SSC2 was paternally expressed with an estimated effect 0.6 mm BFT for the paternally inherited Meishan allele. Under the half-sib model, 12 informative families showed allele substitution effects between 2.1 and 3.9 mm of BFT at the best position across families. Within all these families, the Meishan haplotype was associated with higher BFT. To illustrate the exclusive paternal expression at the tip of SSC2, the tests statistic of four different models are compared in FIG. 7.

For comparative purposes a map of SSC2 loci has been provided with FIG. 9

The QTL on SSC7 mapped to the same region as the QTL affecting TGR and LGR. Like the growth QTL, the effect of the Meishan allele is against expectation because it gives rise to lower BFT (Table 4). For the half-sib analysis, 14 families exceeded the nominal 5% threshold at the overall best position of the QTL, with estimated allele substitution effects between 2.0 and 4.4 mm of BFT. For all these families the Meishan haplotype was associated with lower BFT.

The QTL on SSC14 was maternally expressed with an estimated effect of 0.55 mm of BFT for a maternally inherited Meishan allele. Given the maternally expressed QTL at 30 cM (Table 4), it was surprising that a suggestive QTL was detected in the same marker interval under the (paternal) half-sib analyses.

The QTL on the X chromosome was highly significant with a much larger effect in the F2 males compared to the females. An F2 boar carrying an allele originating from the Meishan had on average 2.5 mm more BFT than a boar carrying an allele originating from the Dutch lines. In the F2 sows, the estimated difference was only 1.5 mm (Table 4).

The strongest suggestive QTL was detected on SSC4 (p=0.13) with an estimated effect of 0.35 mm lower BFT for the Meishan allele, indicating a second cryptic allele. This QTL maps to a different marker interval than the QTL affecting the three growth traits on SSC4 (FIG. 4). On SSC5, a suggestive QTL maps to the same interval as a suggestive QTL affecting LGR. However, the QTL affecting BFT is paternally expressed whereas maternal expression was inferred for the QTL affecting LGR. The suggestive QTL at 91 cM is maternally expressed and maps to a different region than any of the putative QTL affecting the growth traits on SSC6.

3.2. Results for Reproduction Traits

The best QTL affecting litter size are summarized in Table 6. The line-cross analyses revealed one suggestive QTL affecting LS1 and three suggestive QTL affecting LS2. It must be noted that the number of F2 animals with phenotypic data available for these traits is very low compared to the performance traits (Table 3). For LS1, a suggestive QTL was detected on SSC7 with maternal expression. The estimated effect was 1.5 piglet more for a sow inheriting a maternal Meishan allele instead of an allele originating from the Dutch lines. For LS2, three suggestive QTL were detected on SSC12, SSC14, and SSC17. The suggestive QTL on SSC12 was over-dominant with an estimated dominance effect of 1.7 piglet and an estimated additive effect of 0.3 piglet. The suggestive QTL on SSC14 was maternally expressed with an estimated effect of similar magnitude as the suggestive QTL affecting LS 1 on SSC7, but with opposite sign (Table 6). The suggestive QTL on SSC17 showed some slight over-dominance with estimated additive and dominance effects of −0.9 and 1.4 piglet respectively. Because Meishan pigs have on average larger litters than commercial lines, the QTL effects on SSC14 and SSC17 are opposite the expectation, with the Meishan allele giving a lower litter size.

For production traits, a number of genome scans have been performed and many candidate genes have been evaluated. This study has revealed several QTL affecting growth and fat deposition in pigs and added interesting new insight into their mode of expression. Given the large number of QTL identified in the present study, we will not discuss all the identified QTL in detail. In the following paragraphs we will first discuss some findings for specific traits and subsequently interesting results across traits and models.

4.1 Backfat Thickness

For BFT, the pre-adjustment of the trait values was done with body weight at end of test as a covariable. This is conform the analysis of carcass backfat thickness (BF-HGP) by De Koning et al. (1999), where carcass weight was included as a covariable. In the segregation analyses of BFT on the population described in this study, Janss et al. (1997) did not include body weight as a covariable. To test the effect of trait definition, we also analyzed BFT without pre-adjustment for body weight. Under the model without adjustment for body weight, the genome-wide significant QTL affecting BFT on SSC14 was only suggestive while the QTL on SSC2, SSC7, and SSCX remained genome-wide significant albeit with lower test statistics than those reported in Table 4. In contrast, the QTL on SSC4 was genome-wide significant (p=0.01) under the model without adjustment for body weight while it was only suggestive (p=0.13) under the model presented in Table 4. This can be explained by the QTL affecting growth, also on SSC4 (Table 4, FIG. 4). Given the negative effect of the Meishan allele for the QTL on SSC4 for all performance traits, the estimated QTL effect for BFT absorbs part of the effect of the growth QTL on SSC4, when not adjusting for body weight.

The paternally expressed QTL affecting BFT on SSC2 is in line with earlier investigations for QTL affecting BF-HGP on this experimental populations (De Koning et al., 2000; Rattink et al., 2000). However, the position of the imprinted QTL affecting BFT in the present study (5 cM) is different compared to results for BF-HGP (36 cM) and closer to the IGF2 region, for which an imprinted QTL has been reported earlier (Jeon et al., 1999; Nezer et al., 1999). Using only animals that had observations for both ultrasonic and slaughter backfat thickness (n=774) showed that the different position for BFT in the present study could not be attributed to the larger number of animals in the present study (data not shown). The phenotype for slaughter backfat consisted of a single measurement on the carcass (De Koning et al., 1999) whereas the BFT phenotype in the present study was the average of 4-8 measurements at different positions along the spine. The estimated genetic correlation between the two traits was 0.93 using MTDFREML (Boldman et al., 1995), which allows some differences in genes affecting both traits. The test statistic for a Mendelian QTL along SSC2 shows a very broad peak, indicating that, beside the paternally expressed QTL near the IGF2 region, there might be additional QTL affecting BFT on SSC2.

The QTL affecting BFT on SSC7 is in agreement with findings for BF-HGP that were reported in literature and discussed by De Koning et al. (1999).

4.2 Growth

This study provides strong evidence for QTL affecting growth on SSC1. However, there is some variation in the most likely position and the genetic model of the QTL for the different growth traits (Table 4). Also the profiles of the test statistics in FIG. 6 do not point towards a single QTL affecting all three growth traits. Especially the broad peak for LGR suggest that there are multiple QTL affecting growth on SSC1, although a model fitting two Mendelian QTL did not give a significant improvement. Support for additional growth QTL comes from Paszek et al. (1999) who report a QTL affecting growth in a region comparable to our last marker interval on SSC1. One of the candidate genes on SSC1 is the melanocortin-4 receptor (MC4R). To our knowledge, this is the first study reporting a significant QTL affecting growth on SSC8. This is not surprising because the QTL shows exclusive maternal expression. Under a Mendelian model, which is applied in most studies to date, the QTL was only suggestive. The QTL was genome-wide significant for EGR and LGR, suggestive for TGR, and imprinted throughout. It seems that the effect of this QTL is strongest for earlier growth as can also be seen in FIG. 5. It can also be seen from FIG. 5 that the test statistics for EGR and LGR are significant but rather flat. This means that, although there is significant evidence for an imprinted QTL affecting growth on SSC8, its position cannot be estimated very accurately in the present study. A possible explanation could the presence of two or more QTL affecting growth on SSC8.

4.3 Across Traits and Models

4.3.1 Imprinting

In the present study, imprinting was only inferred if a model with only a single parental component was not significantly worse compared to a model with both parental components and a dominance component. For some loci, like the QTL affecting growth on SSC8, this is true for all the examined traits. For other loci, like the growth QTL on SSC4, we inferred an imprinted QTL for TGR and a Mendelian QTL for EGR at similar positions. When looking at the contributions of the three components across a range of traits the classification as Mendelian or imprinted is not that unequivocal. Table 7 shows the F ratios for the three components for a number of traits and SSC4 and SSC7. When looking across traits, there is a range in parent-of-origin effects from Mendelian expression to uni-parental expression rather than a clear division between Mendelian and imprinted QTL. For the QTL affecting EGR on SSC4 all three components are contributing equally (Table 7). The QTL affecting BFT was inferred as Mendelian, although the paternal component is negligible and only the maternal and dominance component are significant (Table 7). The QTL affecting TGR shows only a significant maternal component and the QTL affecting LGR shows the combined action of the QTL affecting EGR and TGR (Table 7). For SSC7 the QTL affecting LGR has comparable contributions for the paternal and maternal component, whereas for BF-HGP, TGR and BFT the paternal component is still highly significant but considerably less extreme than the maternal component. For MD-HGP, the difference between the paternal and maternal component was so extreme that a maternally expressed QTL was inferred (De Koning et al., 2000). This range of parent-of-origin effects provides support for the hypothesis that QTL might be imprinted during specific stages of development and show Mendelian expression at other stages.

4.3.2 Chromosome 4

SSC4 has been a popular chromosome since the first report of a significant QTL affecting growth and backfat thickness in an experimental cross between Wild Boar and Large White by Andersson et al. (1994). The QTL was confirmed within descendants of this experimental population by Marklund et al (1999), while Knott et al. (1998) showed a significant imprinting effect on SSC4 in the same population described by Andersson et al. (1994). Walling et al. (1998) showed significant QTL on SSC4 affecting fatness and growth in an F2 cross between Meishan and Large White. The most likely positions of the QTL were very comparable between Walling et al. (1998) and the present study. Although in both studies the Meishan allele is associated with lower growth, the effect of the Meishan allele on backfat thickness is different. Walling et al (1998) report an increased backfat thickness for the Meishan allele whereas in the present study the Meishan allele gives lower backfat.

Part of the animals of the present study (n=586) were included in a joint analysis for SSC4 of pig data from six countries described by Walling et al. (2000). Although no significant effects were found in the individual analysis of the Dutch data, the direction of the estimated QTL effects were in agreement with the present study (Walling et al., 2000). The results from the present study, illustrated by FIG. 4, and the results from Walling et al. (1998, 2000) point toward multiple linked QTL affecting growth and fatness on SSC4, rather than a single QTL. A possible dissection of these QTL does not only require additional markers but also software that can fit multiple QTL with different genetic models.

4.3.3 Chromosome 6 and Chromosome 7

In the present study six suggestive QTL mapped to different regions of SSC6 with maternal expression (7, 91, and 191 cM), Mendelian expression (30 and 33 cM), or an effect under a paternal half-sib design (191 cM) (Table 4, Table 5). Together with the two significant imprinted QTL affecting intramuscular fat content presented by De Koning et al. (2000), the present study provides further evidence for imprinting on SSC6. However, the new evidence from this study gives no indication for specific regions on SSC6 being either paternally or maternally expressed.

The cryptic allele of the QTL affecting backfat thickness on SSC7 has been reported earlier by De Koning et al (1999, 2000) and is a confirmation of earlier findings by Rohrer and Keele (1998) and Wang et al (1998). The present study also confirms a much more recent finding by Rohrer (2000) of a cryptic allele for growth on SSC7. The cryptic nature of the Meishan allele for both traits and the similar positions of the best QTL for all traits considered point toward a single QTL affecting both growth and backfat thickness. To test this, the correlation of the estimated QTL effects for the individual families under the half-sib analysis can be compared to the expected correlation if a QTL only affects one of the traits (Schrooten et al. submitted). Another alternative would be a multivariate QTL analysis that was recently proposed for an outbred F2 design by Knott and Haley (2000). The beneficial effect of the Meishan allele on both growth and backfat thickness make this QTL an interesting candidate for marker assisted introgression. For this purpose it is not required to distinguish between a single pleiotropic QTL or closely linked QTL.

This study has detected QTL affecting growth and backfat thickness on SSC2, SSC4, SSC7, and SSCX. New significant QTL affecting growth have been detected on SSC1 and SSC8, and a new QTL affecting backfat thickness was detected on SSC14. Furthermore, new genome-wide significant imprinting effects were found for SSC4, SSC8, and SSC14. Several suggestive QTL also showed imprinting effects, but need to be confirmed in other populations or for other traits. So far imprinting has only been studies in a limited number of studies, which hampers the comparison across experiments. The power to detect QTL affecting litter size was low due to the small number of F2 sows with reproduction data.

The invention thus provides for the use of well-defined imprinted and x-linked markers for quality directed crossbreeding in pig breeding programs, of which further specific examples here follow.

Breeding programmes aim at improving the genetic merit of individuals through the selection of parents with superior genetic merit. An important component of each breeding program is the identification of animals with the highest genetic merit that can be used as parents for the next generation. For many years, people have recognised the need for genetic evaluation of animals and today best linear unbiased prediction (BLUP) has become the most widely accepted method for genetic evaluation of domestic livestock. This method relies on the use of phenotypic information and assumes a model with a large number of genes each having a small effect. Recently, molecular genetic techniques have evolved, that can be used to collect more detailed genotypic information on individuals and link this to phenotypic characteristics of an individual to locate quantitative trait loci (QTL) to have a significant influence on the trait. Furthermore, molecular markers can be used to trace which alleles or chromosomal segments a parent has transmitted to its offspring. This created the opportunity for marker assisted selection (MAS) in animal breeding programmes.

The present invention provides here the insight that many major QTL affecting body composition are under control of genomic imprinting, or located on the sex chromosome, which has major implications for the practice of animal breeding. Our results call for a revision of methods for genetic evaluation and breeding scheme design that currently ignore non-Mendelian expression.

Identification of imprinted and X-linked QTL opens new perspectives for crossbreeding, which is common practice in pig breeding. Imprinted genes could further accommodate differentiation between sow lines, which are required to have optimal body composition to support their reproductive performance, and boar lines, which ensure high-quality pork.

In the following paragraphs we will first describe the present situation in pig breeding and subsequently show a novel scenario in which strategic use of the identified imprinted and X-linked QTL allows the final product (slaughter pigs) to be tailored to four different markets, using the same breeding populations.

At present, pig breeding programmes focus on pure-bred breeding for additive genetic progress and crossbreeding for dominance effects (heterosis) in the final breeding product. The genetic evaluations are all based on Mendelian inheritance and ignore information on individual QTL. Generally, selection in sire lines is focused on production and meat quality traits like daily gain, backfat thickness (BF), muscle depth (MD) and intramuscular fat (IMF). Due to the short generation interval, genetic progress is best reachable in these sire lines in particular for traits that can be recorded on selection candidates. Selection for improved carcass quality is hampered by the fact that this information can not be collected directly on the selection candidates but only on relatives. In dam lines, selection is focussed on fertility, growth.and carcass quality traits. Selection on growth and carcass traits in these sow lines is hampered by the negative genetic correlations between fertility and production traits. In addition, there is a conflict between the optimum level of backfat for sows and slaughter pigs. Sows are required to have backfat to support the sow during the period that she is nursing here litter. Slaughter pigs are generally required to have low levels of backfat. As a result, the genetic potential of a slaughter pig for traits like backfat thickness and daily gain is compromised by the maternally inherited alleles. An overview of common crossbreeding schemes using three or four pure-bred lines is given in FIG. 8.

Use of the imprinted and X-linked QTL as provided here in a breeding method according to the invention, offer new ways to use the same pure-bred lines to target a broad range of markets.

For the slaughter pigs we for example define four different markets:

1) The Pork pig: Targeting the European market for fresh and processed pork, this pig should have moderate to fast growth. At the slaughter weight of around 110 kg it should have sufficient muscle depth with a low backfat thickness. The intramuscular fat % can be lower than for the other products, but should not drop below 2%. Pork for the processing plants can be slightly fatter and less muscular than pork for the fresh meat market. The pork market is at the moment the main market, but this might change in the future.

2) The Bacon pig: Targeting the English bacon market, this pig is characterised by fast early growth (EGR), sufficient muscle depth (MD) and high intramuscular fat %. Because of the low slaughter weight (˜80 kg) this animal needs relatively high backfat thickness (BF) at a relatively young age.

3) The Parma pig: Targeting the Italian Parma and Serano ham market, this pig has relatively slow growth and low muscle depth. When it is being slaughtered around 130 kg it should have accumulated a lot of backfat and the meat should have a high level of intramuscular fat %.

4) The Japan pig: Targeting the Japanese meat market this pig should exhibit a very high level of fatness at a relatively young age. Both the IMF and the BF should be higher than for any other market while muscularity should be sufficient.

The desired trait values for all four products are summarised in Table 8. For all markets the invention provides a method for breeding a population of domestic animals, preferably pigs, having desired genetic properties, comprising testing animals to be cross-bred with at least one nucleic acid according to the invention, for the presence of a desired allele of a quantitative trait, allowing for example use of a crossbred sow that has been selected for fertility traits. Preferably the crossbred sow demonstrates a high level of fat deposition that will help her to cope with the large energy demands imposed upon her during the nursing period of the large litters of slaughter pigs.

The molecular tools that will enable the diversification towards the described markets comprise the following QTL:

Growth Traits:

a) SSC4: The QTL affecting Early Growth in a Mendelian fashion and later growth with specific maternal expression. The acronym will be GR4.

b) SSC7: The QTL affecting growth in a standard Mendelian Fashion, at the same locus also a Mendelian effect on backfat thickness and a maternally expressed QTL affecting Muscle depth. Following the most likely position the acronym for this QTL is SLA.

c) SSC8: The maternally expressed QTL affecting mainly Early growth=>Gr8

d) Muscle depth:

e) SSC7=>SLA.

f) Backfat thickness:

g) SSC2: A paternally expressed QTL affect fat deposition=>Bf2.

h) SSC7=>SLA.

i) SSCX: X-linked QTL affecting both IMF and Backfat thickness=>FatX.

j) Intramuscular fat %:

k) SSC6: A maternally expressed QTL at 23 cM=>Im6a.

l) SSC6: A paternally expressed QTL at 117 cM=>Im6b

m) SSCX: FatX.

The purebred lines can be selected for either the high or the low allele of each of the listed QTL. For SLA and FatX it is impossible at this stage to determine whether we are dealing with a single pleiotropic QTL or multiple closely linked QTL. For the moment they will both be treated as single loci and the pleiotropic effects will be as found in the experimental data. This means that for SLA there is one allele that gives lower muscle depth and backfat thickness, but an increased growth. The other allele shows antagonistic effects (i.e. high muscle depth, high backfat thickness and decreased growth). For FatX there is one allele that increases both IMF and BF, and one allele that decreases both traits. Im6a and Im6b are located on the same chromosome. However, due to the large difference in chromosomal position (100 cM), Im6a and Im6b will show no linkage and any combination of effects can be selected for in the pure-bred lines.

Based on the desired properties for the different products as summarised in Table 8, the ideal QTL composition for each pigs for each product was derived. The ideal QTL compositions are presented in Table 9. A breeding scheme to reach these ideal compositions will use molecular typing of selection candidates in each of the pure-bred lines. The pure-bred lines will be selected towards homozygosity for the preferred alleles at the QTL as indicated in Table 9. Once the pure-bred lines have the desired molecular configuration (Table 9) no further molecular typing is required for these QTL.

The basic scheme for the production of slaughter pigs still consists of four purebred lines. Two of these lines are selected for growth and meat characteristics (A&B) and two are selected for reproduction traits (C&D). The new QTL provide a unique handle on the meat and growth characteristics as a result of which sows in lines A and B can also be selected for reproductive capacity and serve as multiplier sows in some alternatives. In lines C and D there is only molecular selection on growth and slaughter traits, the phenotypic selection is entirely for reproduction. The complete mating scheme for all markets is given in Table 9 and will not be discussed in detail. Table 9 describes the combination of lines to be used to meet the requirements of the different markers. By making the different combinations, optimum use can be made of the identified QTL and their modes of inheritance.

The pork and bacon market at present make up the largest export segment for the Dutch pig production. The crossbreeding schemes for these markets follow that of a traditional scheme but the efficiency of the selection schemes is improved by exploiting the information in identified QTL in selection. For the bacon market, the female slaughter pigs will get additional fatness through the QTL on the X chromosome. It is expected that boars, which do not inherit the higher allele, will be castrated and hence have sufficient fatness as slaughter pig.

For the Parma and Japan markets, purebred sows from the sire lines (A and B, respectively) will be used as multiplier sows while crossbred CD boars will be used as terminal sire line (Table 9). This allows transmission of fatness alleles from the lines C and D into the slaughter pigs. A further increase in fatness for the Japanese market is obtained by using a sow from the B line that will transmit her fat allele on the X chromosome to both female and male offspring.

### REFERENCES WITH FURTHER EXAMPLES

Andersson, L., Haley, C. S., Ellegren, H., Knott, S. A., Johansson, M., Andersson, K., Andersson Eklund, L., Edfors Lilja, I., Fredholm, M., Hansson, I., Hakansson, J., Lundstrom, K., 1994. Genetic mapping of quantitative trait loci for growth and fatness in pigs. Science 263, 1771-1774.

Boldman, K. G., Kriese, L. A., Van Vleck, L. D., Van Tasse;, C. P., Kachman, S. D. 1995. A manual for use of MTDFREML [DRAFT]. U.S. Department of Agriculture, Agricultural Research Service, Clay Center, NE, U.S.A.

Churchill, G. A., Doerge, R. W., 1994. Empirical threshold values for quantitative trait mapping. Genetics 138, 963-971.

De Koning, D. J., Janss, L. L., Rattink, A. P., van Oers, P. A. M., de Vries, B. J., Groenen, M. A. M., van der Poel, J. J., de Groot, P. N., Brascamp, E. W., van Arendonk, J. A. M., 1999. Detection of quantitative trait loci for backfat thickness and intramuscular fat content in pigs. Genetics 152, 1679-1690.

De Koning, D. J., Rattink, A. P., Harlizius, B., Van Arendonk, J. A. M., Brascamp, E. W., Groenen, M. A. M., 2000. Genome-wide scan for body composition in pigs reveals important role of imprinting. Proc. Natl. Acad. Sci. U.S.A. 97, 7947-7950.

De Vries, A. G., Kerr, R., Tier, B., Long, T., 1994. Gametic imprinting effects on rate and composition of pig growth. Theor. Appl. Genet. 88:1037-1042.

Haley, C. S., Knott, S. A., Elsen, J. M., 1994. Mapping quantitative trait loci in crosses between outbred lines using least squares. Genetics 136, 1195-1207.

Harlizius, B., Rattink, A. P., De Koning, D. J., Faivre, M., Joosten, R. G., Van Arendonk, J. A. M., Groenen, M. A. M., 2000. The X chromosome harbors quantitative trait loci for backfat thickness and intramuscular fat content in pigs. Mamm. Genome 11, 800-802.

Janss, L. L. G., Van Arendonk, J. A. M., Brascamp, E. W. 1997. Segregation analyses for presence of major genes affecting growth, backfat, and litter size in Dutch Meishan crossbreds. J. Anim. Sci. 75, 2864-2876.

Janss, L. L. G., Thomspon, R., Van Arendonk, J. A. M., 1995. Application of Gibbs sampling for inference in a mixed major gene-polygenic inheritance model in animal populations. Theor. Appl. Gen. 91, 1137-1147.

Jeon, J. T., Carlborg, O., Tornsten, A., Giuffra, E., Amarger, V., Chardon, P., Andersson-Eklund, L., Andersson, K., Hansson, I., Lundstrom, K., Andersson, L., 1999. A paternally expressed QTL affecting skeletal and cardiac muscle mass in pigs maps to the IGF2 locus. Nat. Genet. 21, 157-158.

Kim, K. S., Larsen, N., Short, T., Plastow, G., Rothschild, M. F., 2000. A missense variant of the porcine melanocortin-4 receptor (MC4R) gene is associated with fatness, growth, and feed intake traits. Mamm. Genome. 11, 131-135.

Knott, S. A., Elsen, J. M., Haley, C. S., 1996. Methods for multiple-marker mapping of quantitative trait loci in half-sib populations. Theoretical. and. Applied. Genetics 93, 71-80.

Knott, S. A., Haley, C. S., 2000. Multitrait least squares for quantitative trait loci detection. Genetics. 2000. 156, 899-911.

Knott, S. A., Marklund, L., Haley, C. S., Andersson, K., Davies, W., Ellegren, H., Fredholm, M., Hansson, I., Hoyheim, B., Lundstrom, K., Moller, M., Andersson, L., 1998. Multiple marker mapping of quantitative trait loci in a cross between outbred wild boar and large white pigs. Genetics 149, 1069-1080.

Lander, E. S., Kruglyak, L., 1995. Genetic dissection of complex traits: Guidelines for interpreting and reporting linkage results. Nat. Genet. 11, 241-247.

Marklund, L., Nystroem, P., Stern, S., Andersson Eklund, L., Andersson, L., 1999. Confirmed quantitative trait loci for fatness and growth on pig chromosome 4. Heredity 82, 134-141.

Nezer, C., Moreau, L., Brouwers, B., Coppieters, W., Detilleux, J., Hanset, R., Karim, L., Kvasz, A., Leroy, P., Georges, M., 1999. An imprinted QTL with major effect on muscle mass and fat deposition maps to the IGF2 locus in pigs. Nat. Genet. 21, 155-156.

Paszek, A. A., Wilkie, P. J., Flickinger, G. H., Rohrer, G. A., Alexander, L. J., Beattie, C. W., Schook, L. B., 1999. Interval mapping of growth in divergent swine cross. Mamm. Genome 10, 117-122.

Rattink, A. P., De Koning, D. J., Faivre, M., Harlizius, B., Van Arendonk, J. A. M., Groenen, M. A. M., 2000. Fine mapping and imprinting analysis for fatness trait QTLs in pigs. Mammalian. Genome 11, 656-661.

Rohrer, G. A., 2000. Identification of quantitative trait loci affecting birth characters and accumulation of backfat and weight in a Meishan-White Composite resource population. J. Anim. Sci. 78, 2547-2553.

Rohrer, G. A., Ford, J. J., Wise, T. H., Vallet, J. L., Christenson, R. K., 1999. Identification of quantitative trait loci affecting female reproductive traits in a multigeneration Meishan-White composite swine population. J. Anim. Sci. 77, 1385-1391.

Rohrer, G. A., Keele, J. W., 1998. Identification of quantitative trait loci affecting carcass composition in swine: I. Fat deposition traits. J. Anim. Sci. 76, 2247-2254.

Rothschild, M., Jacobson, C., Vaske, D., Tuggle, C., Wang, L., Short, T., Eckardt, G., Sasaki, S., Vincent, A., McLaren, D., Southwood, O., van der Steen, H., Mileham, A., Plastow, G., 1996. The estrogen receptor locus is associated with a major gene influencing litter size in pigs. Proc. Natl. Acad. Sci. U.S.A. 93, 201-205.

Walling, G. A., Archibald, A. L., Cattermole, J. A., Downing, A. C., Finlayson, H. A., Nicholson, D., Visscher, P. M., Walker, C. S., Haley, C. S. 1998. Mapping of quantitative trait loci on porcine chromosome 4. Anim. Genet. 29, 415-424.

Walling, G. A., Visscher, P. M., Andersson, L., Rothschild, M. F., Wang, L., Moser, G., Groenen, M. A., Bidanel, J. P., Cepica, S., Archibald, A. L., Geldermann, H., De Koning, D. J., Milan, D., Haley, C. S., 2000. Combined analyses of data from quantitative trait loci mapping studies. Chromosome 4 effects on porcine growth and fatness. Genetics. 155, 1369-1378.

Wang, L., Yu, T. P., Tuggle, C. K., Liu, H. C., Rothschild, M. F., 1998. A directed search for quantitative trait loci on chromosomes 4 and 7 in pigs. J. Anim. Sci. 76, 2560-2567.

### FIGURE LEGENDS

FIG. 1 Test statistic profiles for three porcine chromosomes that exhibit imprinting effects for one of the body composition traits. The black line represents the test statistic for a Mendelian QTL vs. H₀ of no QTL. The blue line represents the test statistic for a paternally expressed QTL vs. no QTL. The red line represents the test statistic for a maternally expressed QTL vs. no QTL. The black horizontal line denotes the 5% genomewise threshold for the Mendelian model and the blue line indicates the same threshold for the imprinting model. a, SSC2 and backfat thickness; b, SSC6 and intramuscular fat content; c, SSC7 and muscle depth; d, SSC7 and backfat thickness. Homologous human regions are indicated as colored bars (refs. 6, 7, 8 and http://www.toulouse.inra.fr/lgc/pig/cyto/cyto.htm). Imprinted genes located within these human chromosomal areas are underneath. Alignment of the porcine cytogenetic and genetic map is adapted from http://sol.marc.usda.gov/genome/swine/htmls/chromosome_list.html.

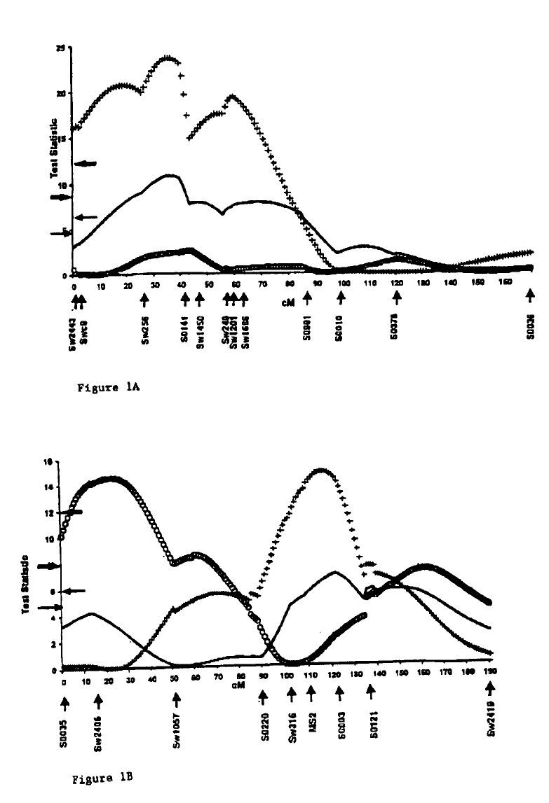

FIG. 2. Test statistic profile for chromosome X. BFT is indicated as a solid line, IMF is shown as a circled line. 5% genomewise significant threshold determined by permutation is shown as a dotted line.

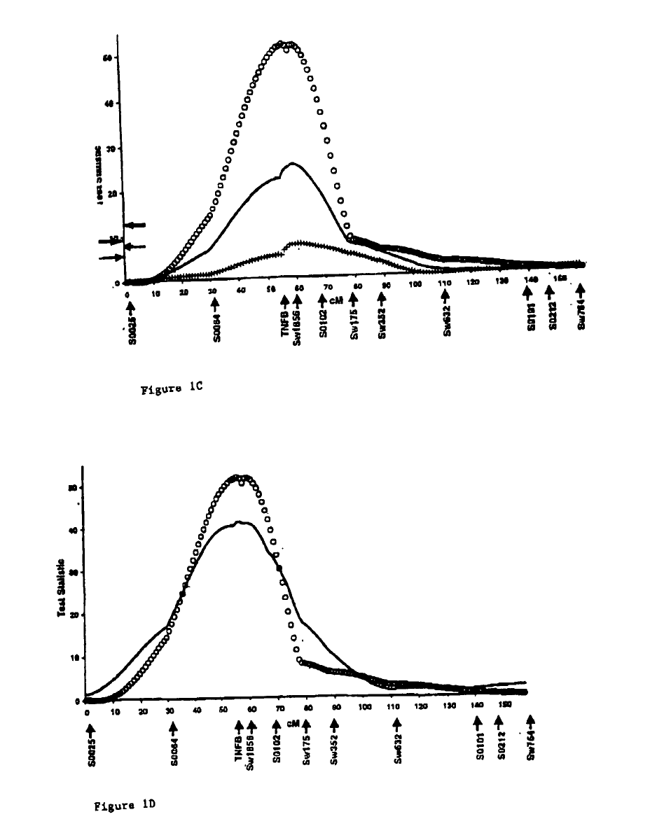

FIG. 3A A 4 breed crossbreeding programme and use of parental imprinting

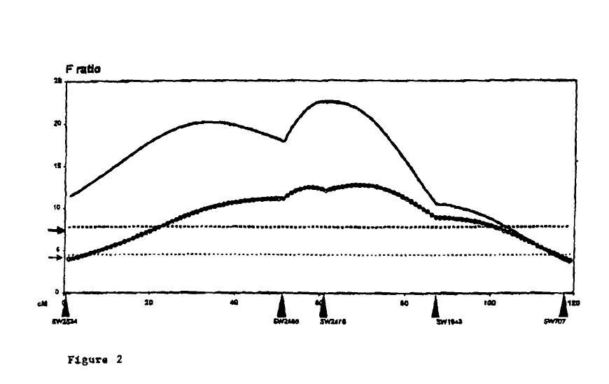

FIG. 3B A 3 breed crossbreeding programme and use of parental imprinting

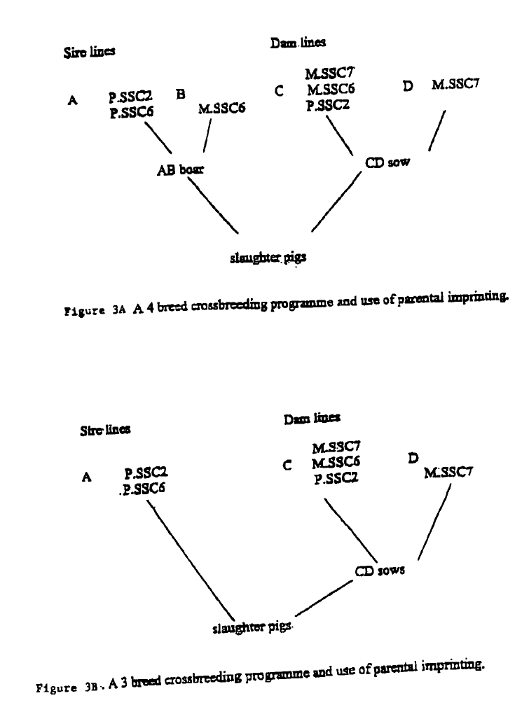

FIG. 4. Test-statistic along SSC4 for QTL affecting Early growth (EGR), Test growth (TGR), Life growth (LGR), or ultrasonic backfat thickness (BFT) for the inferred genetic models. The dashed and solid horizontal lines denote the thresholds for suggestive and genome-wide significant linkage, respectively. Marker names and positions are given above the graph.

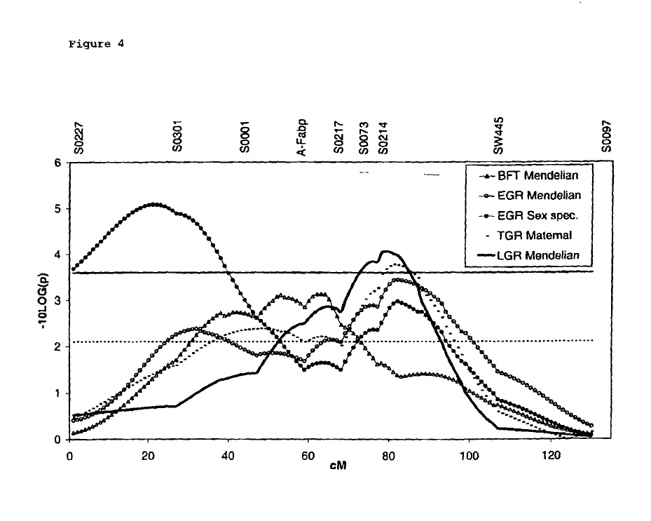

FIG. 5. Test-statistic along SSC8 for the maternally expressed QTL affecting Early growth (EGR), Test growth (TGR), and Life growth (LGR). The test statistic of a putative paternally expressed QTL affecting early growth is included for comparison. The dashed and solid horizontal lines denote the thresholds for suggestive and genome-wide significant linkage, respectively. Marker names and positions are given above the graph.

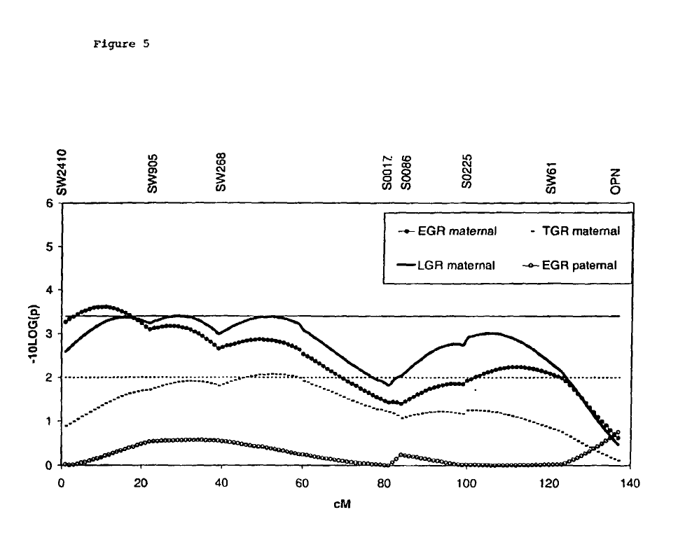

FIG. 6. Test-statistic along SSC1 for QTL affecting Early growth (EGR), Test growth (TGR), or Life growth (LGR) for the inferred genetic models. The dashed and solid horizontal lines denote the thresholds for suggestive and genome-wide significant linkage, respectively. Marker names and positions are given above the graph.

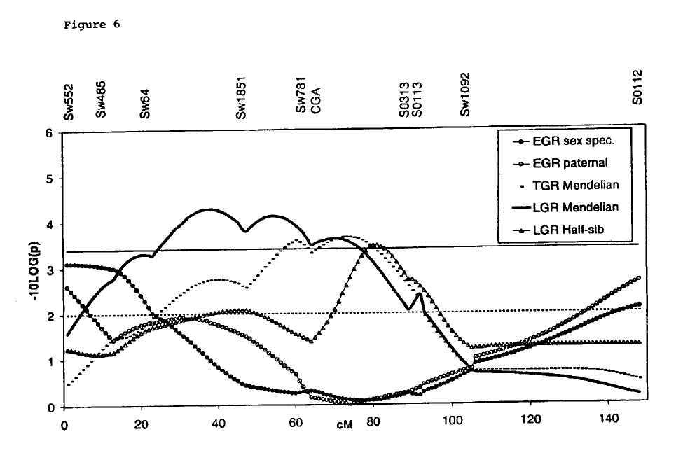

FIG. 7. Test-statistic along SSC2 for the presence of a QTL affecting ultrasonic backfat thickness under four different models. The dashed and solid horizontal lines denote the thresholds for suggestive and genome-wide significant linkage, respectively. Marker names and positions are given above the graph.

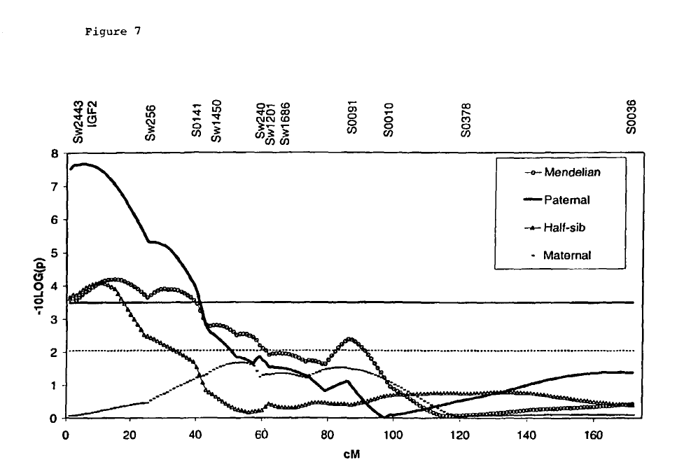

FIG. 8. Common crossbreeding schemes in traditional pig breeding using crosses between three (bottom) or four (top) pure-bred lines. Note that within every pure-bred line sufficient numbers of boars and sows are available to allow sufficient selection intensity.

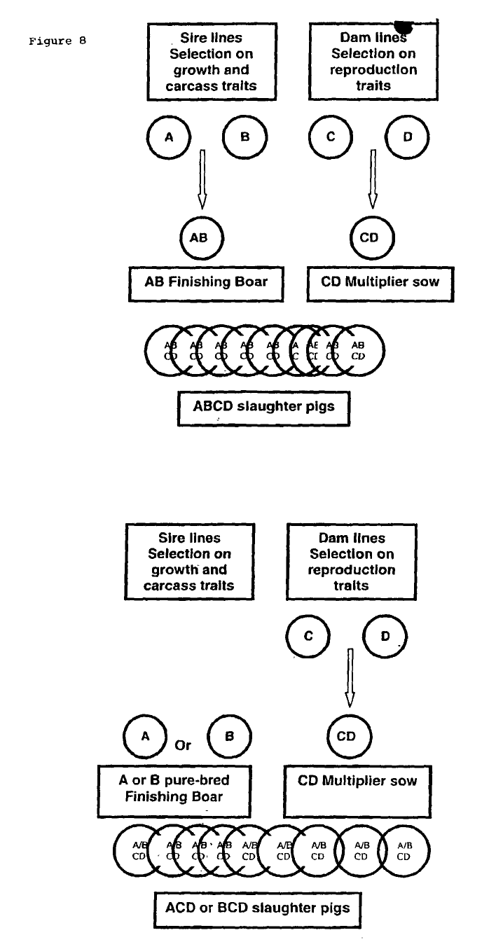

FIG. 9. A comprehensive radiation hybrid map for SSC2 with 58 loci. Framework markers are in bold, whose order is supported by linkage at LOD 4.0. The lengths of linkage groups are indicated in centirays (cR₃₀₀₀) under the last marker of the linkage group. The distances between the linkage groups are, from group I to group V, 102.3, 124.8, 150.6, and 114.2 cR₃₀₀₀. On the right of the RH map of SSC2 are the human radiation position (GB4: http://www.ncbi.nlm.nih.gov/genemap_{99/}, cR), human cytogenetic location, and murine linkage position (cM) of the genes mapped on the RH panel.

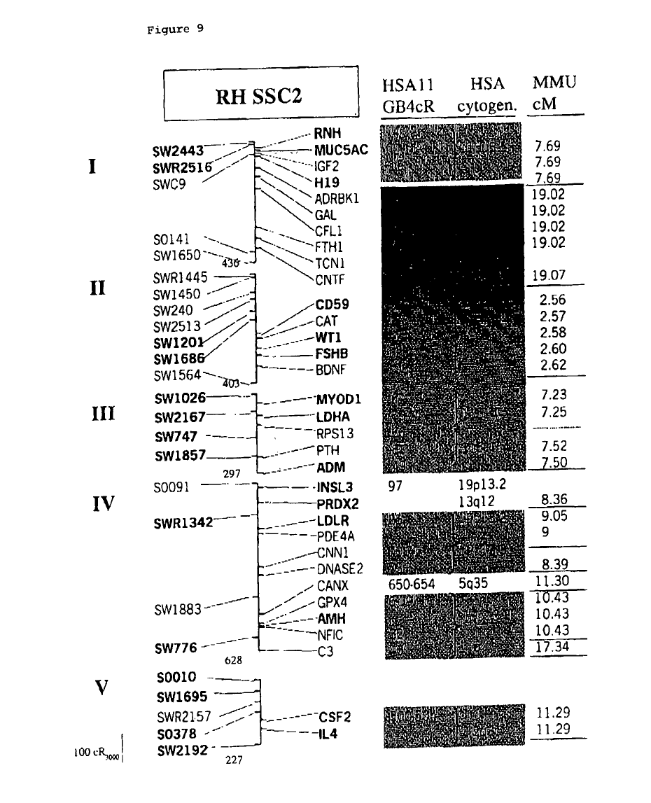

TABLE 1
QTL analysis for three body compostion traits under different genetic models

| | | | | | |
|---|---|---|---|---|---|
| | Genetic | Test | | Confidence | |
| Chromosome | model^{a} | statistic^{b} | Position^{c} | interval^{d} | QTL effect (s.e.)^{e} |

Backfat thickness mm.

| | | | | | |
|---|---|---|---|---|---|
| 2 | Mendelian | 10.72** | 36 | 12-82 | 1.28(0.28) − 0.34(0.43) |
| 2 | Paternal | 23.51*** | 36 | 0-73 | 0.94(0.19) |
| 2 | Maternal | 2.47 | 44 | N/A | 0.30(0.19) |
| 7 | Mendelial | 41.05*** | 57 | 38-72 | −2.30(0.25) − 0.09(0.39) |
| 7 | Paternal | 33.11*** | 61 | N/A | −1.09(0.19) |
| 7 | Maternal | 51.31*** | 56 | N/A | −1.35(0.19) |

Muscle depth mm.

| | | | | | |
|---|---|---|---|---|---|
| 7 | Mendelian | 25.00*** | 59 | 40-70 | −2.21(0.32) − 0.85(0.50) |
| 7 | Paternal | 7.36 | 61 | N/A | −0.66(0.24) |
| 7 | Maternal | 51.46*** | 56 | 41-72 | −1.71(0.24) |

Intramuscular fat
content %

| | | | | | |
|---|---|---|---|---|---|
| 6 | Mendelian | 6.90 | 122 | 101-165 | −0.17(0.05) − 0.01(0.07) |
| 6 | Paternal | 14.68* | 117 | 90-150 | −0.13(0.03) |
| 6 | Maternal | 14.50* | 23 | 0-52 | 0.14(0.04) |

*p < 0.05.
***p < 0.01.
***p < 0.001
^{a}Mendelian refers to a model where an additive and a dominance effect are estimated⁴; Paternal and Maternal refer to models where only a paternally or a maternally expressed QTL effect are estimated.
^{b}Test statistics for the different models versus the H0 of no QTL. The risk levels are on a genomewide level and estimated by permutations⁴,
^{c}Most likely position of a QTL in Haldance cM.
^{d}Empurical confidence intervals obtained by bootstrapping. From each of 10,000 bootstrap replicates the best test statistic was stored. The 95% cut-off point of the sorted (in descending order) test statistics provides an empirical threshold for the confidence interval. Confidence intervals were calculated for the standard model and for the imprinting models when relevant.
^{e}Estimates of additive and dominance effect under the standard model and of the parentally expressed allele under the imprinting models. The additive and the parental effect are expressed as the deviation of the Meishan allele. The dominance effect is expressed as the deviation of the heterozygotes from the mean of the two groups of homozygotes⁴. Standard errors of the estimates are given in parentheses.

TABLE 2
QTL analysis for backfat thickness
and intramuscular fat content on chromosome X.
Test statistics, QTL position and estimated
QTL effects for sexes separately.

| | | positon | Additive effect | |
|---|---|---|---|---|
| Marker bracket | Test statistic | cM | male | female |

Backfat thickness (mm)

| | | | | |
|---|---|---|---|---|
| SW2456/SW2476 | 22.56*** | 60 | 1.47 ± 0.249 | 1.02 ± 0.321 |

Intramuscular fat content (%)

| | | | | |
|---|---|---|---|---|
| SW2476/SW1943 | 12.79*** | 69 | 0.21 ± 0.046 | 0.13 ± 0.060 |

***p < 0.001, test statistic not exceeded in 10.000 permutations according to (Churchill and Doerge, 1994). Table 3. Measured traits with number of F2 animals with observation (N_{fen}), number of F2 animals included in QTL analyses (N_{QTL}), phenotypic means and standard deviations. Adapted from Janss et al. (1997).

| | Full name, measurement | | | | |
|---|---|---|---|---|---|
| Trait | unit | N_{fen} | N_{QTL} | Mean^{a} | SE^{a} |
| EGR | Early growth, g/day | 1020 | 942 | 448.3 | 79.1 |
| TGR | Test growth, g/day | 1020 | 942 | 657.0 | 126.5 |
| LGR | Life growth, g/day | 1246 | 1151 | 522.6 | 76.2 |
| BFT | Ultrasonic backfat | 1218 | 1131 | 15.6 | 3.8 |
| | thickness, mm | | | | |
| LS1 | Litter size (total number | 269 | 249 | 11.0 | 3.2 |
| | born) at first farrowing | | | | |
| LS2 | Litter size (total number | 222 | 206 | 11.7 | 3.2 |
| | born) at second farrowing | | | | |

^{a}For all the animals that have phenotypic information

TABLE 4
Location and characterization of QTL affecting performance
traits that exceed suggestive linkage under the line-cross models

| | Marker [bracket] | Genetic | Test statistic | | |
|---|---|---|---|---|---|
| Chromosome | (position, cM) | Model^{a} | (p)^{b} | a (s.e.)^{c} | D (s.e.)^{c} |
| EGR (9/day) | | | | | |
| SSC1 | SW552-SW485 (2) | Sex specific | 4.79 (0.06) | | |
| | | | | −19.0 (5.5) | −9.5 (7.3) |
| | | | | −12.1 (6.0) | 4.4 (7.9) |
| SSC1 | S0112 (148) | Paternal | 9.45 (0.21) | 16.7 (5.4) | — |
| SSC4 | S0227-S0301 (21) | Sex specific | 7.32 (<0.001) | | |
| | | | | 11.6 (6.0) | 16.2 (9.0) |
| | | | | −28.2 (6.9) | 27.3 (9.4) |
| SSC4 | S0214 (82) | Mendelian | 8.00 (0.07) | −12.6 (3.8) | 14.3 (5.8) |
| SSC5 | SWR453-SW332 (30) | Mendelian | 5.38 (0.46) | 0.8 (4.6) | 26.0 (7.9) |
| SSC6 | SW2419 (191) | Maternal | 11.81 (0.07) | −9.6 (2.8) | — |
| SSC8 | SW2410-SW905 (10) | Maternal | 13.54 (0.03) | −11.5 (3.1) | — |
| SSC8 | SW905-SW268 (23) | Sex specific | 3.74 (0.47) | | |
| | | | | −12.7 (5.5) | −6.1 (7.5) |
| | | | | −12.4 (5.9) | 11.5 (8.0) |
| SSC10 | SW1041 (85) | Paternal | 10.93 (0.07) | −9.0 (2.7) | — |
| SSC13 | S0076-S0068 (43) | Maternal | 6.38 (0.61) | −10.3 (4.1) | — |
| TGR (g/day) | | | | | |
| SSC1 | CGA-S0313 (73) | Mendelian | 8.5 (0.03) | −26.2 (6.4) | −1.6 (10.6) |
| SSC2 | SW256 (25) | Paternal | 7.14 (0.57) | −13.7 (6.2) | — |
| SSC4 | S0073-S0214 (81) | Maternal | 14.26 (0.02) | −15.5 (4.1) | — |
| SSC6 | SW2406-SW1057 (31) | Mendelian | 5.83 (0.31) | −20.7 (7.3) | 9.5 (9.1) |
| SSC7 | SW1856 (59) | Mendelian | 29.70 (<0.001) | 40.8 (5.5) | 15.8 (8.4) |
| SSC8 | SW268-S0017 (53) | Maternal | 6.98 (0.56) | −15.3 (5.8) | — |
| SSC12 | S0090-S0106 (79) | Paternal | 7.39 (0.43) | −11.6 (4.3) | — |
| SSC13 | S0076-S0068 (67) | Mendelian | 6.54 (0.17) | −1.9 (7.8) | 57.1 (15.8) |
| SSC14 | SW210-S0007 (65) | Paternal | 7.36 (0.43) | −13.3 (4.9) | — |
| LGR (g/day) | | | | | |
| SSC1 | SW64-SW1851 (38) | Mendelian | 9.9 (0.007) | −16.1 (3.7) | −5.9 (6.3) |
| SSC3 | SW72 (1) | Paternal | 9.11 (0.26) | −6.9 (2.3) | — |
| SSC3 | S0206-SW902 (20) | Mendelian | 6.07 (0.28) | −11.2 (3.4) | −8.3 (5.4) |
| SSC4 | S0073-S0214 (79) | Mendelian | 9.42 (0.02) | −12.8 (3.3) | 11.1 (5.1) |
| SSC5 | S0005-IGF1 (80) | Maternal | 6.74 (0.64) | −7.4 (2.8) | — |
| SSC6 | SW2406-SW1057 (33) | Mendelian | 6.53 (0.17) | −12.5 (4.2) | 17.7 (8.1) |
| SSC6 | S0035-SW2406 (7) | Maternal | 6.72 (0.57) | −6.8 (2.6) | — |
| SSC7 | SW1856 (59) | Mendelian | 35.01 (<0.001) | 22.2 (3.1) | 18.7 (4.7) |
| SSC8 | SW905-SW268 (29) | Maternal | 12.63 (0.07) | −9.6 (2.7) | — |
| SSC13 | S0076-S0068 (72) | Mendelian | 5.03 (0.53) | 0.6 (4.3) | 27.1 (8.5) |
| SSC17 | SWR1004 (1) | Maternal | 8.56 (0.40) | −6.5 (2.2) | — |
| BFT (mm) | | | | | |
| SSC2 | IGF2-SW256 (5) | Paternal | 31.2 (<0.001) | 0.61 (0.11) | — |
| SSC4 | AFabp-S0217 (63) | Mendelian | 7.25 (0.13) | −0.35 (0.15) | −0.69 (0.23) |
| SSC5 | S0005-IGF1 (83) | Paternal | 9.67 (0.18) | 0.37 (0.12) | — |
| SSC6 | S0220-SW316 (91) | Maternal | 8.43 (0.30) | 0.33 (0.12) | — |
| SSC7 | SW1856-S0102 (63) | Mendelian | 57.86 (<0.001) | −1.5 (0.14) | 0.17 (0.22) |
| SSC14 | SW857-SW295 (30) | Maternal | 13.65 (0.02) | 0.55 (0.15) | — |
| SSCX | SW2456-SW2467 (58) | X-linked | 43.66 (<0.001) | | — |
| | | | | 1.28 (0.16) | |
| | | | | 0.77 (0.15) | |

^{a}Genetic model which is most appropriate for the QTL.
^{b}Test statistics for the inferred genetic model vs. the H0 of no QTL, p values are genome-wide significance levels.
^{c}Estimated QTL effects for the inferred genetic model. The additive effect a is expressed as the deviation of the Meishan allele and the dominance effect d is expressed (where appropriate) as the deviation of the heterozygous animals from the mean of the homozygotes.

TABLE 5
Location of QTL affecting performance traits that exceed
suggestive linkage under the half-sib model

| | | Marker [bracket] | |
|---|---|---|---|
| Trait | Chromosome | (position, cM)^{a} | Test statistic (p)^{b} |
| TGR | SSC7 | LTA (55) | 2.02 (0.04) |
| LGR | SSC1 | CGA-S0313 (81) | 2.01 (0.03) |
| LGR | SSC3 | SW72 (1) | 1.74 (0.38) |
| LGR | SSC6 | SW2419 (191) | 1.67 (0.46) |
| LGR | SSC7 | SW1856-S0102 (61) | 2.85 (<0.001) |
| BFT | SSC2 | IGF2-SW256 (10) | 2.15 (0.02) |
| BFT | SSC7 | SW1856-S0102 (61) | 2.89 (<0.001) |
| BFT | SSC14 | SW857 (1) | 1.67 (0.63) |

^{a}Best position across families.
^{b}Test statistic across families for the H0 of no QTL, p values are genome-wide significance levels

TABLE 6
Location and characterization of QTL affecting litter size that exceed
suggestive linkage under the line-cross models

| | | | | | |
|---|---|---|---|---|---|
| | Marker [bracket] | Genetic | Test statistic | | |
| Chromosome | (position, cM) | Model^{a} | (p)^{b} | a (s.e.)^{c} | d (s.e.)^{c} |

LS1 total number born

| | | | | | |
|---|---|---|---|---|---|
| SSC7 | S0025-S0064 (10) | Maternal | 8.73 (0.38) | 0.75 (0.25) | — |

LS2 total number born

| | | | | | |
|---|---|---|---|---|---|
| SSC12 | S0090 (71) | Mendelian | 5.92 (0.26) | 0.29 (0.32) | 1.70 (0.51) |
| SSC14 | SW210-S0007 (62) | Maternal | 7.20 (0.51) | −0.75 (0.28) | — |
| SSC17 | SW840-SW1031 (43) | Mendelian | 5.35 (0.61) | −0.91 (0.39) | 1.39 (0.66) |

^{a}Genetic model which is most appropriate for the QTL.
^{b}Test statistics for the inferred genetic model vs. the H0 of no QTL, p values are genome-wide significance levels.
^{c}Estimated QTL effects for the inferred genetic model. The additive effect a is expressed as the deviation of the Meishan allele and the dominance effect d is expressed (where appropriate) as the deviation of the heterozygous animals from the mean of the homozygotes.

TABLE 7
Contributions of the paternal, maternal, and
dominance components for the QTL on SSC4 and SSC7.
The QTL are ordered with increasing degree
of imprinting within a linkage group.

| | | F ratios for individual | | |
|---|---|---|---|---|
| | | components of the model | | |
| Trait | Position | Paternal | Maternal | Dominance |
| EGR | SSC4 82 cM | 5.29 | 5.81 | 6.00 |
| LGR | SSC4 79 cM | 5.08 | 10.65 | 4.75 |
| BFT | SSC4 63 cM | 0.24 | 7.67 | 8.30 |
| TGR | SSC4 81 cM | 2.23 | 15.16 | 2.27 |
| LGR | SSC7 59 cM | 22.13 | 28.60 | 15.92 |
| BF-HGP^{†} | SSC7 57 cM | 30.27 | 49.35 | 0.04 |
| TGR | SSC7 59 cM | 18.14 | 36.37 | 3.66 |
| BFT | SSC7 63 cM | 36.19 | 81.02 | 0.49 |
| MD-HGP^{‡} | SSC7 56 cM | 4.74 | 50.33 | 2.20 |

^{†}Backfat thickness, measured with Hennessy Grading probe.
^{‡}Muscle depth, measured with Hennessy Grading probe.

TABLE 8
Desired properties of slaughter pigs for four
different markets, standardised to a weight of 100 kg.

| | Pork | Bacon* | Parma | Japan* |
|---|---|---|---|---|
| Growth g/day | 825 | 850 | 750 | 800 |
| Backfat thickness | 15 | 20 | 24 | 28 |
| mm | | | | |
| Muscle depth mm | 59 | 57 | 40 | 57 |
| Intramuscular fat % | 2 | 3 | 3 | 4 |
| Slaughter weight | 110 | 75 | 130 | 80 |
| (kg) | | | | |

*Animals will be slaughtered <100 kg so actual values of backfat and muscle depth will be lower.

Table 9. Molecular composition of four pure-bred lines, n crossbred types and four types of slaughter pigs. + Indicates selection or expression for the high allele of a QTL, − selection or expression for the low allele of a QTL and 0 indicates no selection or expression for that QTL in the specific line and a “?” indicates that the inherited allel can be either + or −. For the crossbred animals and the Mendelian QTL in the slaughter pigs both parental alleles are given (first is paternally inherited).

| | Boar x | | | | | | | |
|---|---|---|---|---|---|---|---|---|
| | Sow | Gr4 | GR8 | Bf2 | Im6a | Im6b | SLA* | FatX |
| A | Pure | + | − | − | − | − | +−− | − |
| B | Pure | + | + | − | + | + | −++ | + |
| C | Pure | + | + | + | + | + | −++ | − |
| D | Pure | + | + | + | + | + | −++ | − |

F1 multipliers

| | | | | | | | | |
|---|---|---|---|---|---|---|---|---|
| AB | A × B | +/+ | −/+ | −/− | −/+ | −/+ | +−−/−++ | |
| | | | | | | | | +−/+ |
| CD | C × D | +/+ | +/+ | +/+ | +/+ | +/+ | −++/−++ | |
| | | | | | | | | −−/− |

Slaughter pigs

| | | | | | | | | |
|---|---|---|---|---|---|---|---|---|
| Pork | A × | +/+ | + | − | + | − | +−0/−++ | |
| | CD | | | | | | | −−/− |
| Bacon | AB × | +/+ | + | − | + | ? | ?/−++ | |
| | CD | | | | | | | −+/− |
| Parma | CD × A | +/+ | − | + | − | + | −+0/+−− | |
| | | | | | | | | −−/− |
| Japan | CD × B | +/+ | + | + | + | + | −+0/−++ | |
| | | | | | | | | ++/− |

*+−−indicates the maternally inherited allele with higher growth and lower backfat thickness and muscle depth, while −++indicates the antagonistic allele. +−0 and −+0 are the equivalents of the paternally inherited alleles for this locus, which will have no effect on muscularity.

What is claimed is:

1. An isolated and/or recombinant nucleic acid comprising a sequence affecting the quantitative trait muscle depth in mammals, said sequence being derived from a locus corresponding to region HSA6p_{21.3}-p.22 in humans and corresponding to the homologous region on ssc7 in pigs.

2. The isolated and/or recombinant nucleic acid of claim 1, wherein said quantitative trait is maternally expressed.

3. An isolated and/or recombinant nucleic acid comprising a sequence affecting the quantitative trait back fat thickness in mammals, said sequence being derived from a locus corresponding to a region on ssc7 in pigs, which region overlaps with the region HSA6p_{21.3}-p.22 in humans and corresponding to the homologous region on ssc7 in pigs affecting the quantitative trait muscle depth on ssc7 in pigs.

4. An isolated and/or recombinant nucleic acid comprising a sequence affecting the quantitative trait back fat thickness in mammals, said sequence being derived from a locus corresponding to a region on ssc2 of pigs which maps about 35 cM away from the IGF2 region.

5. The isolated and/or recombinant nucleic acid of claim 4, wherein said quantitative trait is paternally expressed.

6. An isolated and/or recombinant nucleic acid comprising a sequence affecting the paternally imprinted quantitative trait intramuscular fat in mammals, said sequence being derived from a locus corresponding to a region on the long arm of ssc6 of pigs.

7. An isolated and/or recombinant nucleic acid comprising a sequence affecting the maternally expressed quantitative trait intramuscular fat in mammals, said sequence being derived from a region HSA6q22-ter in humans and corresponding to the homologous on the short arm of ssc6 of pigs.

8. An isolated and/or recombinant nucleic acid comprising a sequence affecting the quantitative trait intramuscular fat in mammals, said sequence being derived from a region localized at about 60 cM on the X chromosome in pigs.

9. An isolated and/or recombinant nucleic acid comprising a sequence affecting the quantitative trait back fat thickness in mammals, said sequence being derived from a region localized at about 69 cM on the X chromosome in pigs.

10. The isolated and/or recombinant nucleic acid of any one of claims 1-9 which is a specific probe or primer for a quantitative trait.

11. An isolated and/or recombinant nucleic acid comprising a sequence capable of identifying a quantitative trait as identified in any one of claims 1-9.

12. The isolated and/or recombinant nucleic acid of claim 10 or claim 11, comprising a microsatellite marker.

13. A method of identifying a quantitative trait in a mammal comprising using the isolated and/or recombinant nucleic acid of any one of claims 1-12 to identify a quantitative trait locus in the mammal.

14. The method according to claim 13, wherein said quantitative trait locus is subject to genomic imprinting.

15. A method for selecting a domestic animal for desired genetic properties comprising using the isolated and/or recombinant nucleic acid of any one of claims 1-12 to select the domestic animal having desired the genetic properties.

16. The method according to claim 15, wherein said domestic animal is a breeding animal or an animal for slaughter.

17. A method for breeding a population of domestic animals having desired genetic properties, said method comprising:
testing animals to be cross-bred with at least one nucleic acid of any one of claims 1-12, for the presence of a desired allele of a quantitative trait.

18. The method according to claim 17, wherein said testing comprises contacting a sample from an animal to be cross-bred with a nucleic acid according to any one of claims 1-12 and detecting the presence or absence of hybridization.

19. A mammalian cell comprising the recombinant nucleic acid of any one of claims 1-9.

20. A transgenic animal comprising at least one cell of claim 19.
